(19) United States
(12) Patent Application Publication
(10) Pub. No.: US 2014/0199758 A1
(43) Pub. Date: Jul. 17, 2014

(54) BILIRUBIN HEMATOLFLUOROMETER AND REAGENT KIT

(71) Applicant: Aviv Biomedical, Inc., Lakewood, NJ (US)

(72) Inventors: Jack Aviv, Lakewood, NJ (US); Angelo A. Lamola, Vallejo, CA (US); Glen Ramsay, Toms River, NJ (US); Jeff MacDonald, Little Egg Harbor, NJ (US)

(73) Assignee: AVIV BIOMEDICAL, INC., Lakewood, NJ (US)

(21) Appl. No.: 14/154,647

(22) Filed: Jan. 14, 2014

Related U.S. Application Data
(60) Provisional application No. 61/752,540, filed on Jan. 15, 2013.

(51) Int. Cl. G01N 33/72

(52) U.S. Cl. CPC G01N 33/728
USPC 435/288.7; 422/82.08; 422/514

(57) ABSTRACT

A reagent kit for detecting bilirubin in a fluid sample. The reagent kit includes a body defining at least one fluid receiving well and an optical window positioned over each at least one fluid receiving well. Each window is formed of a material having a fluorescence intensity that is of a lower magnitude than the fluorescence to be detected from the bilirubin. A hematofluorometer configured to utilize the reagent kit is also disclosed.

This application claims the benefit of U.S. Provisional Application No. 61/752,540, filed on Jan. 15, 2013, the contents of which are incorporated herein by reference.

### FIELD OF THE INVENTION

This invention relates to a device and methods for determining the level of bilirubin and the bilirubin binding status in a blood sample from a patient. More particularly, the invention relates to a bilirubin hematofluorometer and reagent kits for use therewith.

### BACKGROUND OF THE INVENTION

Bilirubin is processed in our bodies by the enzyme glucuronosyl transferase so that it can be excreted. In about half of all neonates, upregulation of this enzyme is delayed, and bilirubin accumulates to levels that may cause neurological damage, including a condition known as kernicterus. Jaundice is a symptom of bilirubin accumulation. When a jaundiced infant is diagnosed, the baby may be promptly given blue light phototherapy (bilirubin is converted by the light into more excretable forms) and the baby stays in the hospital until the bilirubin level is deemed safe. The level of bilirubin deemed safe is, in current practice, determined by a complicated set of “rules” that involve several clinical parameters. It is often, especially in premature infants, difficult to discern whether an infant requires an exchange transfusion, the slower acting phototherapy, or not immediate treatment for the jaundice.

With hospitals now sending newborns home within 24 hours, infants may not develop jaundice or other signs of kernicterus until after they are sent home. As such, those infants may not receive the prompt treatment they need, and neurological damage affecting cognitive, auditory and motor skills may result.

### SUMMARY OF THE INVENTION

Briefly, the present invention provides a hematofluorometer with algorithms for processing fluorescence intensity signals as a function of the temperature and the hemoglobin content or hematocrit of the sample.

In another aspect, the invention provides a reagent kit with one or more wells configured to receive a blood sample and one or more reagents. Each well has a corresponding window which is designed to not interfere adversely with the relevant florescence signal from the sample.

The assays that can be performed using the hematofluorometer provide information about the risk for adverse effects of bilirubin in each particular infant. Such information has been shown to be useful in managing jaundiced infants but has been difficult to obtain by other means. The hematofluorometer assays have been shown to be extremely easy to perform and require only a couple of drops of blood that can be obtained from a “heel stick.” The assays would be useful in managing sick neonates in the intensive care nursery, to manage discharged infants upon return to the outpatient clinic or pediatrician office, and to assay the capability of an infant to safely handle becoming jaundiced should they become jaundiced after being discharged.

More specifically, today there are needs for an inexpensive, easy-to-use, portable (battery powered) system for the assay of plasma bilirubin and bilirubin binding status at the point-of-care of neonates with hyperbilirubinemia. Ideally, the system would require less than 100-microliters of blood such as can be readily obtained by “heel stick” and require minimal manipulation of the blood specimen. There are at least three different populations that would benefit from such a system: the neonate in the intensive care nursery, the neonatal outpatient in developed countries, and the jaundiced neonate in underdeveloped countries.

It has been the trend in developed countries for several years now that apparently healthy neonates, even including moderately low-birth weight babies, are discharged from hospital within a day or two from birth. And unless there is some indication of jaundice, there is no pre-discharge blood bilirubin assay. These neonates are generally followed by means of return visits to an outpatient clinic or by means of a visiting nurse at home. This practice has reduced health care costs because of reduced hospital stay but has complicated the management of jaundice once it appears in the discharged neonate. There is evidence that concomitant with this early discharge practice there has been an increase in the incidence of kernicterus and neurological sequelae. The system described herein allows for point-of care assays by a visiting nurse at home or by a pediatrician in the outpatient clinic or private office. Eliminating the need for blood drawing in sufficient quantity for transport to the clinical laboratory and time delay in awaiting the results, will both facilitate treatment decisions and minimize time to action if necessary. Given an inexpensive system, this approach could also reduce cost substantially.

Alternatives to the system described herein are the transcutaneous bilirubinometers (reflectance measurements through the skin) and some stat wet chemical bilirubin assays using small instruments. While the transcutaneous bilirubinometers have been found useful for following the trend in bilirubin level they have not been widely accepted because of variability depending on skin color, site of measurement, and operator skill. The instruments and disposables are expensive. The stat wet chemical methods that work best require separation of the plasma from the blood and are not amenable to visiting nurse or pediatrician desk use. In any case, neither approach can give information regarding bilirubin binding status.

The idea of a pre-discharge bilirubin assay is controversial simply because, depending on skin color, the test result would generally be found unremarkable in the first few hours after birth in the absence of a visual observance of jaundice. Two aspects of the system described herein can change the view of a pre-discharge assay. The overall benefit of a pre-discharge blood bilirubin assay should be evident given a simple enough, low blood volume, and inexpensive enough approach such as described here. Probably more valuable than a bilirubin assay is the total binding capacity for bilirubin. There is a large body of published work indicating that only when the bilirubin level in the blood approaches half or more of the quantity of albumin capable of binding the bilirubin does the risk for neurological effects becomes high. Those neonates for whom a lower than optimal capacity is found could then be given a higher priority for careful follow-up should jaundice appear. There would be less concern for those neonates with normal binding capacity. Presently there is no point-of-care system available for bilirubin binding status.

Care of the sick and or premature and low birth weight neonate in the hospital is complicated. The determination of treatment modality for such infants when they are jaundiced is based upon a decision tree recommended by the American Academy of Pediatrics and is based on clinical experience using parameters such as the rate of increase in bilirubin level, gestational age, and birth weight. It is for this population that a stat and low volume method for the bilirubin binding status would be useful as an additional guide in judging therapy options and progress for that particular neonate. There exists no stat method for bilirubin binding today. The most examined method, the so called “peroxidase” method is a cumbersome laboratory-bound method. The fluorescence approach described herein has been shown to give results in agreement with the “peroxidase” method.

In the underdeveloped world, where neonatal jaundice is unappreciated for the extent of mortality and morbidity it affects, having a battery-powered portable and very inexpensive system to assay bilirubin in blood by itinerant health care personnel could bring dramatic improvement.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a schematic view of a hematofluorometer in accordance with a first exemplary embodiment of the invention.

FIG. 2 is a plan view of an exemplary reagent kit.

FIG. 3 is a cross-sectional view along the line 3-3 in FIG. 2.

FIG. 4 is a plan view of an alternative exemplary reagent kit.

FIG. 5 is a cross-sectional view along the line 5-5 in FIG. 4.

FIG. 6 is a plan view of another alternative exemplary reagent kit.

FIG. 7 is a plan view of yet another alternative exemplary reagent kit.

FIG. 8 is a side elevation view of the reagent kit of FIG. 7.

FIGS. 9-11 are top views of the reagent kit of FIG. 7 illustrating sequentially filling of the well thereof.

FIGS. 12 and 13 are cross-sectional views of another exemplary reagent kit.

FIGS. 14-17 are cross-sectional views of alternative exemplary caps useable with the various reagent kits.

FIG. 18 is a schematic view similar to FIG. 1 illustrating the reagent kit of FIG. 12 or 13 positioned relative to the hematofluorometer.

FIG. 19 is a schematic view of another exemplary hematofluorometer in accordance with the invention.

FIG. 20 is a side elevation view of a pipette of an alternative embodiment of the reagent kit.

FIG. 21 is a perspective view of the alternative embodiment of the reagent kit with a fluid sample being loaded from the pipette to a glass slide of the kit.

### DETAILED DESCRIPTION OF THE INVENTION

In the drawings, like numerals indicate like elements throughout. Certain terminology is used herein for convenience only and is not to be taken as a limitation on the present invention. The following describes preferred embodiments of the present invention. However, it should be understood, based on this disclosure, that the invention is not limited by the preferred embodiments described herein.

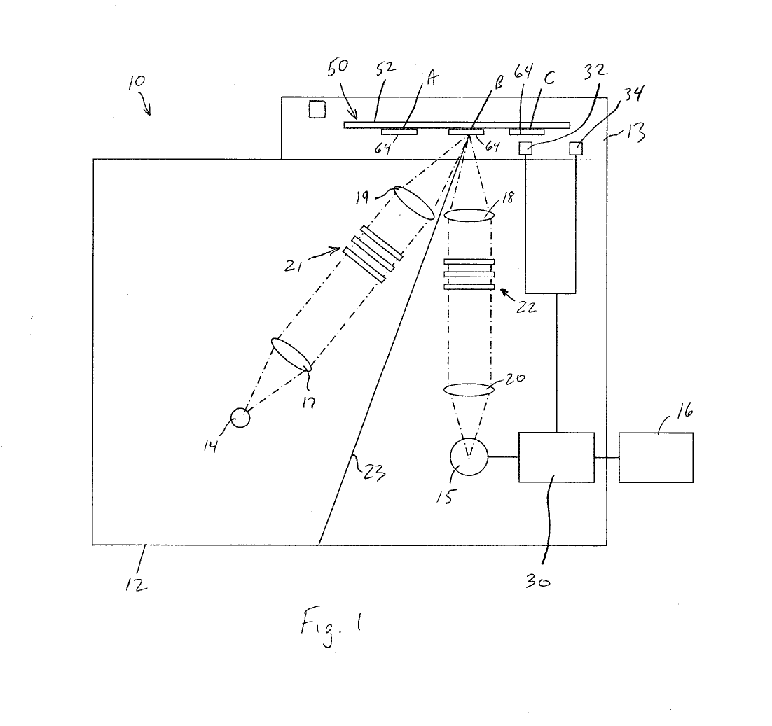

An exemplary hematofluorometer 10 is illustrated in FIG. 1 and generally includes a major housing 12, a minor housing 13 for receiving samples, an excitation source 14, a fluorescence detector 15 and display means 16. Other elements schematically depicted include excitation beam collimating means 17, fluorescence emission collimating means 18, excitation focusing means 19, fluorescence focusing means 20, wavelength band narrowing means 21 and 22 and partition 23. These elements are similar to those described in U.S. Pat. No. 3,973,129 which is incorporated herein by reference. The hematofluorometer 10 can be powered by a rechargeable battery (not shown) and can also be powered by house AC via an appropriate transformer.

The means 17 through 20 are preferably simple lenses, while means 21 and 22 are preferably filter packs which may be fixed or changeable optical filters. In either event the minimum requirement is for elimination of the long wavelength portion of excitation from source 14 to prevent overlap with the fluorescence to be detected by means 15. Preferred design for optical filter packs results in a specifically defined band pass at each of the two positions. Means 21 results in a defined band corresponding with a suitable absorption region in the sample to be studied while means 22 results in a similarly well-defined region centered about the fluorescence wavelength of concern. It is contemplated that either or both of means 21 and 22 may consist of or include more specific elements, such as, gratings, prisms, or adjustable interference filters and may include one or more polarizing elements. Alternatively, fiber optics (fiber bundles) in near field may be used to bring excitation light to the specimen and emitted fluorescence to the detector. The optical measurements can include absorbance, fluorescence, scattering, or any other method involving light and small quantities of sample and other fluids. It is also contemplated to include light in two different wavelengths, for example, green for the hemoglobin determination and blue for the bilirubin determinations.

Referring to FIG. 1, the hematofluorometer 10 of the present embodiment includes a central processing unit (CPU) 30 connected to one or more sensors 32 and one or more input devices 34 and to the fluorescence detector 15 and the display means 16. While illustrated as separate components, one or more of the components, for example the display means 16 and the input device 34, may be integral components.

The one or more sensors 32 are configured to sense one or more of the following variables: dark Intensity (IGlass); reference intensity (IRer); unprocessed blood intensity (IUB); bilirubin saturated intensity (IBS); and temperature (T). Additional variables may also be measured and utilized for calibration depending on the specific application. Exemplary values which may be entered using the input device 34 include the percent hematocrit (PHct) or hemoglobin (Hb). In a preferred embodiment, the units of PHct will be a percentage in the range of 20 to 70% and the Hb will be in units of g/dL with a range of 7.0 to 23.0.

The temperature measurement can be used to correct for the temperature dependent response of the instrument, as well as temperature dependent changes in the fluorescence of the fluorescent bilirubin, and temperature dependent changes in the equilibrium of bilirubin binding. Similarly, the CPU 30 may make calculations for a correction of the hematocrit or hemoglobin content of the blood. The hematocrit is known to affect the fluorescence measurement by affecting the depth of penetration of the light into the sample. Furthermore, the hematocrit, being the volume of the sample that is occupied by blood cells, is a necessary value for use in converting blood concentration to serum concentration to conform to current clinical usage.

In the illustrated embodiment, the sensed values or input values will be provided to the CPU 30. The CPU 30 may be programmed with additional information to assist in calibration of the instrument. For example, the CPU 30 may have values stored for hemoglobin to hematocrit conversion; conversion from intensity to concentration (c); enthalpy change (dH); entropy change (dS); dark offset; dissociation constant (c′). The system may be set with default values, preferably which can be adjusted by the user. The CPU 30 may be provided with additional constants, for example, the free energy change (dG); the binding constant (K); and the temperature corrected conversion from intensity to concentration (c′). Preferably these values may also be adjusted by the user to give correct output values

Upon completion of calculations, the CPU 30 will send desired calculated values to the display means 16. The displayed calculated values may include bound bilirubin (B) (mg/dL serum); binding capacity (C) (mg/dL serum); reserve binding capacity: (R) (units will be milligrams per deciliter of serum, mg/dL); bound/reserve ratio (B/R or B/(C−B)); saturation index (no units); and temperature: T (Celsius).

Under an exemplary procedure, the user will provide two samples for measurement, one with unprocessed blood and another with blood that is saturated with bilirubin. The fluorescent intensity of these samples, the dark, reference and temperature will be measured. As part of the process, the user will have the option of entering in an Hb value or PHct value of the blood sample or opting for no entry, for example, because only the B/R ratio is desired.

The CPU 30 process the data utilizing the following algorithms:

Conversion from hemoglobin to hematocrit fraction is:

Hct=h*Hb (1)

Conversion from percent hematocrit to hematocrit (this is only needed if the convention is to express the hematocrit as a percentage):

Hct=PHct/100 (2)

Intensity values corrected for dark offset, dark and reference values:

I_{UB′}=(I_{UB}+I_{DO}−I_{D})/(I_{Ref}+I_{DO}−I_{D}) (3)

I_{BS′}=(I_{BS}+I_{DO}−I_{D})/(I_{Ref}+I_{DO}−I_{D}) (4)

Calculation of the bound bilirubin concentration present in the plasma is:

B=c I_{UB′}hct/(1−hct) (5)

Calculation of the total binding capacity is:

C=c I_{BS′}hct/(1−hct) (6)

The above equations do not take into account corrections for temperature effects. The correction for the change in quantum yield is:

I″=I′ 10^{0.0128 (T-25)} (7)

Where T is the temperature in Celsius and the reference temperature is 25° C. The constant of 0.0128 is derived from the data presented in “Fluorometric Study of the Partition of Bilirubin among Blood Components: Basis for Rapid Microassays of Bilirubin and Bilirubin Binding Capacity in Whole Blood” (1979) Angelo A. Lamola, Josef Eisienger, William E. Blumberg, Samantha C. Patel, Jorge Flores, Analytical Biochemistry V100: 25-42, incorporated herein by reference. With this correction the equations 5 and 6 can be rewritten as:

B=c I_{UB′}(10^{0.0128 (T-25)})hct/(1−hct) (8)

C=c I_{BS′}(10^{0.0128 (T-25)})hct/(1−hct) (9)

The temperature correction for the change in binding constant can then be calculated.

Calculation reserve binding capacity is:

R=C−B (11)

The ratio of bound/reserve is informative as a measure of unbound or free bilirubin:

B/R (12)

The saturation index can also be used as a measure of the unbound or free bilirubin:

Saturation Index=10×B/R (13)

The ratio of B/R multiplied by the dissociation constant is the unbound bilirubin level (“U”):

U=c′(B/R) (14)

But c′ is also temp dependent because B/R and U are related by the binding constant which is temperature dependent

The sensed temperature can also be used as a check for whether the instrument is too cold or hot to make accurate measurements. Provided the temperature is within a desired range, the device 10 can run the test at the sample temperature and the CPU 30 applies a temperature correction to the calculation. The same temperature reading is used to correct for the effect of temperature on instrument response. Fluorescence intensity values measured vary with the temperature of the device, because, among other things, of the effect of temperature on photomultiplier tube performance. The software uses the same temperature measurement to correct for temperature-dependent variations in device performance.

Referring again to FIG. 1, one or more samples (A-C) are positional relative to the excitation source 14 by positioning a reagent kit 50 within the minor housing 13. In the illustrated embodiment, the minor housing 13 houses a vibration mechanism 80, for example, an eccentric rotatably mounted weight. The vibration mechanism 80 may be controlled by the CPU 80 to vibrate and thereby shake a reagent kit 50 positioned within the minor housing 13 to initiate or maintain mixing of the samples.

The system makes use of the principles of hematofluorometry, that is, fluorescence measurements made on whole blood using excitation wavelengths so strongly absorbed by the hemoglobin that even thin blood samples are optically dense (OD>2). This means that the fluorescence has to be observed in the so-called “front face” mode wherein the excitation impinges upon and the fluorescence observed emanates from the same surface of the specimen. The minor housing 13 is configured to maintain the reagent kits 50 in such an orientation.

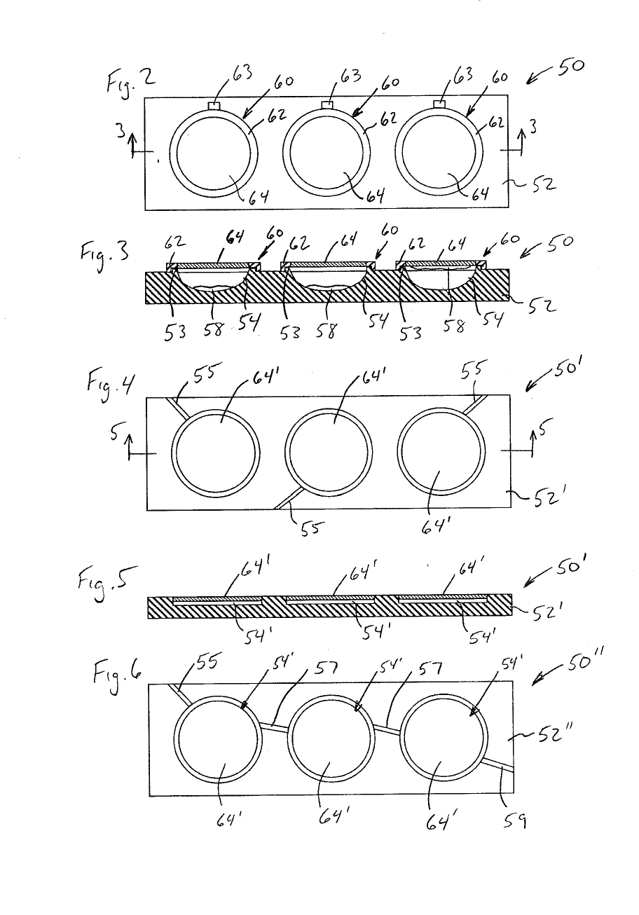

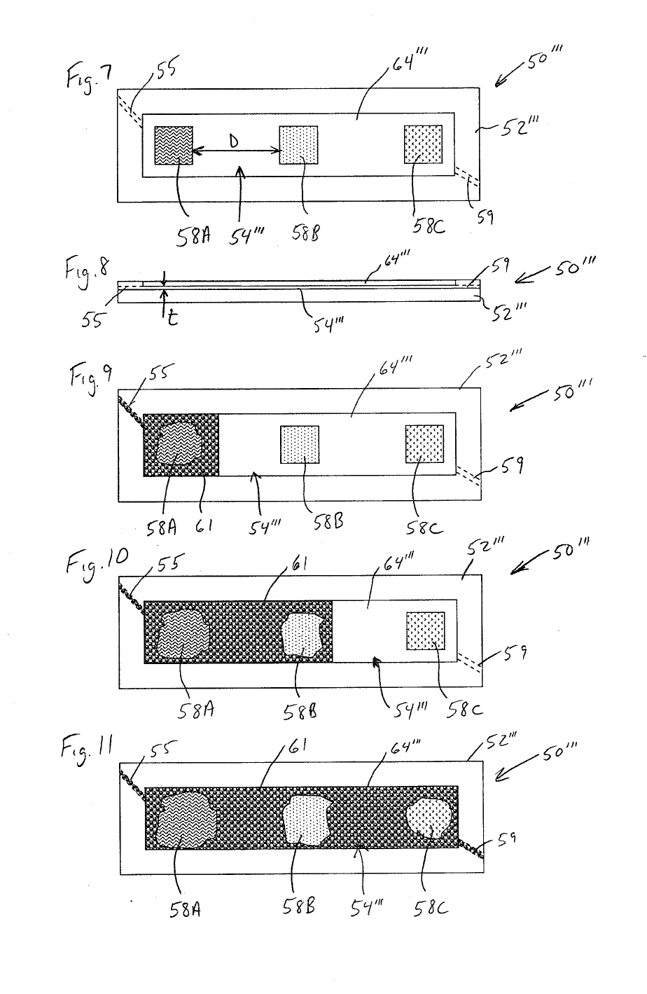

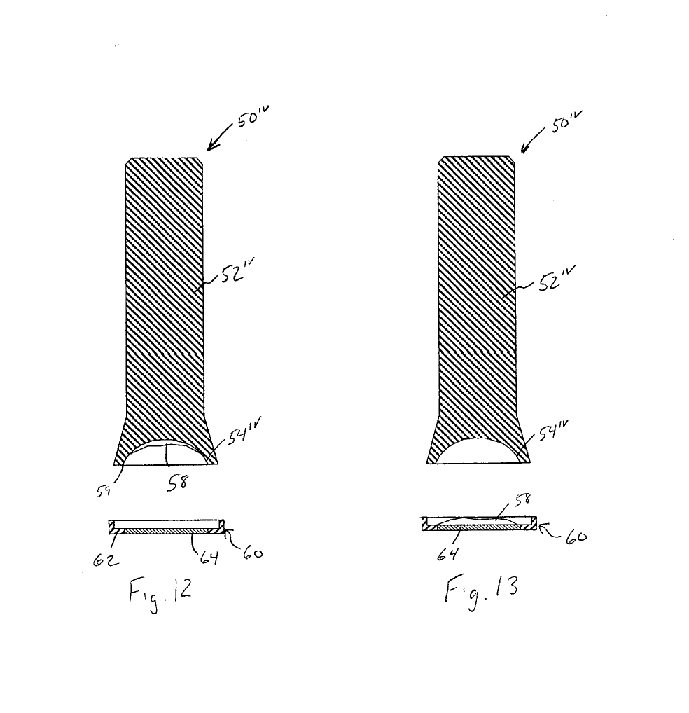

Exemplary reagent kits 50 will be described with reference to FIGS. 2-17. Referring to FIGS. 2 and 3, a first exemplary reagent kit 50 is shown. The reagent kit includes a body 52 defining a plurality of spaced apart wells 54. A respective rim 53 extends from the upper surface of the body 52 about each well 54. The rims 53 are configured to engage with corresponding caps 60. Each cap 60 has an outer rim 62 which sealingly engages a respective rim 53. A hinge 63 may extend between the body 52 and the outer rim 62 to facilitate hinged opening of the caps 60. A central portion of each cap 60 defines a window 64 which facilitates passage of the excitation beam without auto-fluorescence. In a preferred embodiment, the window 64 is made from silica glass, however, other materials which do not cause excessive auto-fluorescence can be used. Exemplary materials include Zeonex™ 48R resin, cyclicolefin copolymers such as Topas™ 8007 X 10 and other such materials available from Ticona Corp or Zeon Chemicals; and polymethylpentene based plastics available from Mitsui. It is also possible to use materials that have intrinsic fluorescence if the fluorescence intensity is reasonably constant and of a lower magnitude than the fluorescence to be detected from the bilirubin since it would then be possible to correct for the background fluorescence without introducing unacceptable error in the bilirubin assay. The remainder of the body 52 and the caps 60 may be manufactured from moldable polymers or the like, for example, PMMA, polystyrene and polyolefins. Having multiple wells 54 and corresponding windows 64 allows multiple samples to presented and analyzed with a single reagent kit 50. The positions may be distinguished by functionality (such as mode of measurement), chemistry, or may simply be redundant to allow repeated measurements.

Referring to FIG. 3, each well 54 has an associated reagent 58. The reagents 58 are preferably dried on either the surface of the well 54 or on the cap 60. Reagents 58 could include a large variety of items, including but not limited to: ligands, surfactants, buffers, salts, reactants, anti-clotting agents, antibodies, dyes, fluorophores or any other type of materials that have any type of desired effect on the sample. When the sample is provided in the well 54 and the cap closed, the reagents 58 would dissolve into the sample. Shaking or other mixing means may be utilized to assist with dissolving of the reagents 58. The appropriate reagent 58 and sample quantities and ratios can be maintained for accurate measurements by the volume of the well 54 such that it only receives the appropriate amount of sample.

Referring to FIGS. 4 and 5, an alternative exemplary reagent kit 50′ will be described. The reagent kit 50′ is similar to the previous embodiment and includes a body 52′ with a plurality of wells 54′ defined therein. In the reagent kit 50′ of the present embodiment, the windows 64′ are secured within the body 52′ above respective wells 54′ without the needs for caps. As in the previous embodiment, a reagent (not shown) is provided in each well 54′. To facilitate filling, each well 54′ has a respective capillary inlet 55 which extends from the well 54″ to an outer edge of the body 52′. The inlet 55 is positioned relative to a sample fluid and capillary action draws the fluid into the well 54′. Once filled, the reagent kit 50′ is utilized in the manner described above.

Referring to FIG. 6, the reagent kit 50″ is similar to the previous embodiment, however, instead of each well 54′ having its own capillary inlet, the body 52″ includes a single capillary inlet 55. The adjacent wells 54′ are connected to one another via intermediate capillary passages 57 and a pressure equalizing passage 59 extends from the last well 54′. The inlet 55 is positioned relative to a sample fluid and capillary actions draws fluid into all of the wells 54′.

Referring to FIGS. 7-11, another exemplary reagent kit 50″′ will be described. The reagent kit 50″′ is similar to the previous two embodiments in that it utilizes capillary action to fill the reagent kit. In the present embodiment, a single laterally extending well 54″′ is defined in the body 52″′ and is covered by a single window 64″′. As seen in FIG. 7, three different reagents 58A, 58B and 58C are provided within the well 54″′, spaced from one another by a distance D. The distance D is significantly greater than the depth t of the well 54″′ from the window 64″′. A capillary inlet 55 and outlet 59 are in communication with the well 54″′.

FIGS. 9-11 illustrate the filling of the reagent kit 50″′. As can be seen, because the well depth t is significantly less than the distance D between reagents 58A and 58B, the reagent 58A will dissolve through the thin layer of fluid 61 and present at the window 64″′ relatively quickly compared to the amount of time it will take for the reagent 58A to dissolve over the distance D to interfere with reagent 58B. The same occurs for reagent 58B compared to reagent 58C. As such, each reagent 58A, 58B, 58C defines a sample area for a sufficient time for measuring.

Referring to FIGS. 12-18, another exemplary reagent kit 50^{iv} will be described. The reagent kit 50^{iv} has a tubular body 52^{iv} with an open cup at one end which defines the well 54^{iv}. The well 54^{iv} is generally of a small size such that when an edge 59 thereof is positioned relative to a fluid sample, the fluid will fill the well 54^{iv} based on capillary action. A cap 60 having an outer rim 62 and a central window 64 is configured to sealingly close the open cup well 54^{iv}. A reagent 58 may be provided on the inside surface of the well 54^{iv} or on the inside surface of the cap 60. After the well 54^{iv} is filled, the cap 60 is secured in position and the fluid sample is mixed with the reagent 58. The cap 60 may be tethered to the body 52^{iv} if desired. FIG. 18 illustrates how the reagent kit 50^{iv} may be positioned relative to the device 10 with the window 64 presenting the sample.

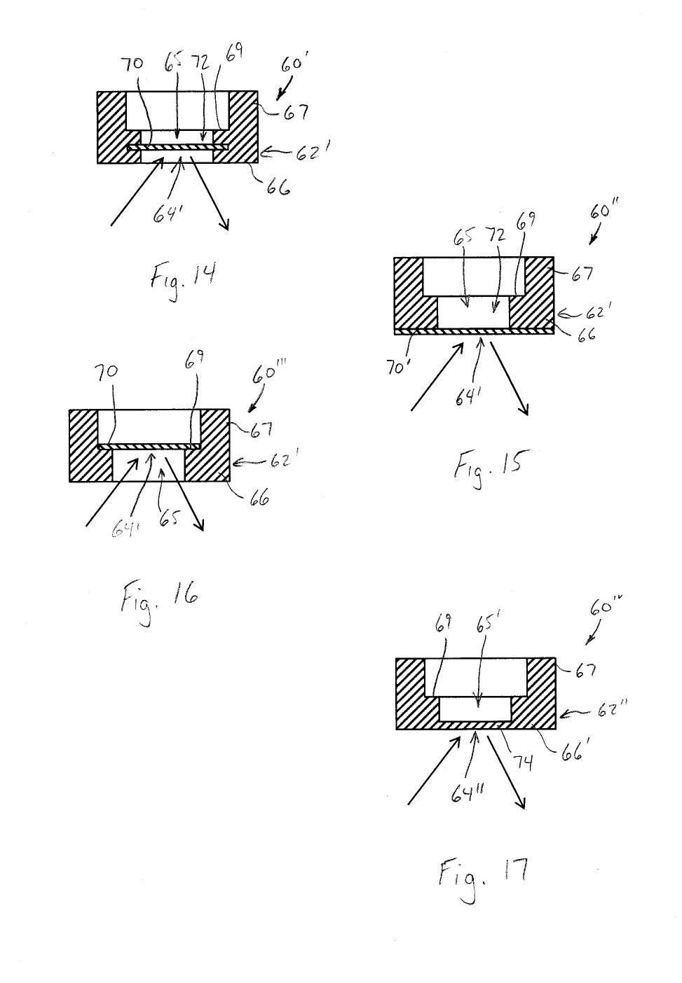

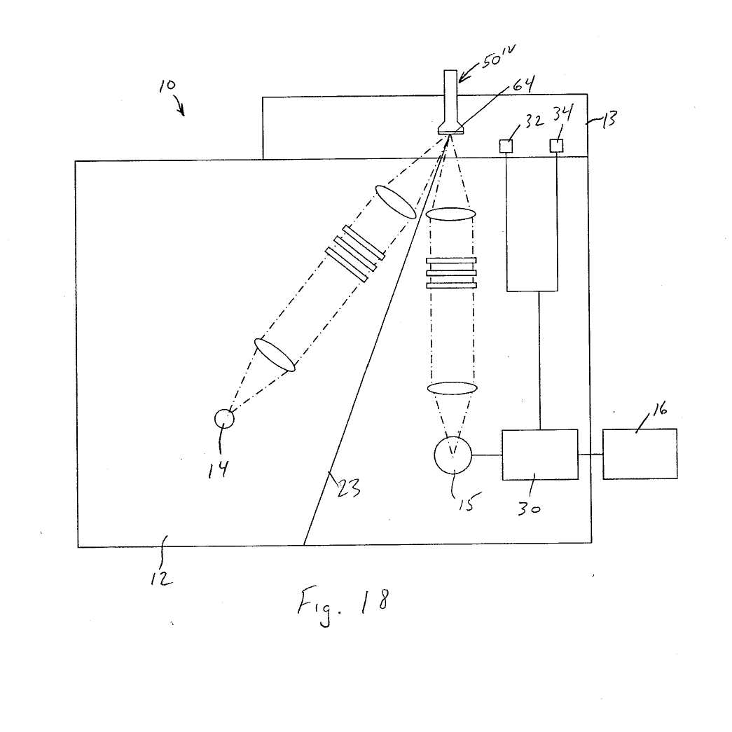

Referring to FIGS. 14-17, various caps that may be utilized with the reagent kit 50^{iv}, as well as others of the above-described kits, will be described. FIG. 14 illustrates a cap 60′ having an outer rim 62′ and a central window 64′ The outer rim 62′ includes a planar portion 66 and a depending portion 67 with an internal shoulder 69 defined therebetween. A central through passage 65 extends through the planar portion 66 with a plate 70 extending thereacross to define the window 64′. The plate 70 is manufactured from silica glass or other materials which do not cause excessive auto-fluorescence. Exemplary materials include Zeonex™ 48R resin, cyclicolefin copolymers such as Topas™ 8007 X 10 and other such materials available from Ticona Corp or Zeon Chemicals; and polymethylpentene based plastics available from Mitsui. The plate 70 in the present embodiment is spaced from the shoulder 69 such that a fluid sample receiving cavity 72 is defined at the window 64′. As the cap 60′ is positioned on the body 52^{iv}, the depending portion 67 and shoulder 69 guide the fluid sample into the cavity 72 and the shoulder 69 defines a stop such that the appropriate volume of sample is positioned at the window 64′.

The caps 60″ and 60″′ in FIGS. 15 and 16 are similar to the cap 60′ except for the position of the plate 70, 70′. Referring to FIG. 15, the plate 70′ of cap 60″ is positioned along the outer surface of the planar portion 66 such that the cavity 72 has a maximum depth relative to the shoulder 69. The plate 70 may be in the form of a film that extends across all or part of the outer surface of the planar portion. With reference to FIG. 16, the plate 70 is supported on the shoulder 69 such that the cap 60′″ does not define a receiving cavity and the cavity will be limited to the volume defined by well 54^{iv} of the tubular body 52^{iv}.

The cap 60^{iv} illustrated in FIG. 17 is similar to the cap 60′ except that the passage 65′ does not extend completely through the planar portion 66′ of the rim 62″ and a remaining portion 74 of the planar portion 66′ defines the window 64″. The remaining portion 74 preferably is a generally thin portion, for example, with a thickness of approximately 0.1 to 0.2 mm. In this embodiment, the entire cap 60^{iv} is preferably manufactured from a material which does not cause excessive auto-fluorescence.

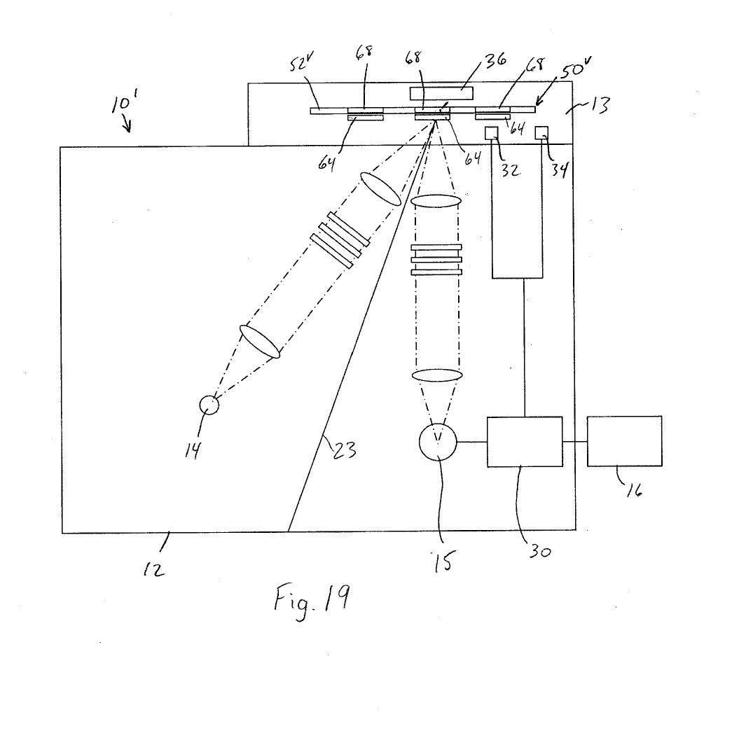

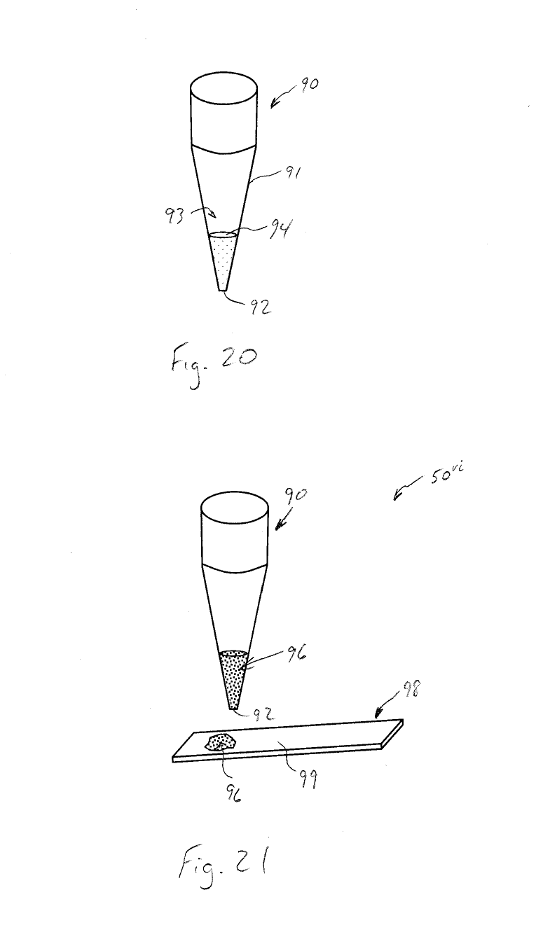

Referring to FIG. 19, another exemplary hematofluorometer 10′ is shown. The device 10′ is similar to the previous embodiment, however, the minor housing 13 includes an additional light sensor 36 on the back side of the reagent kit position. Each window 64 or reagent kit 50^{v} has a corresponding window 68 out the rear of the body 52^{v} such that light passing through the window 64 will continue through the window 68 to the sensor 36. The intensity of the transmitted light could be used to measure sample light absorbance. This absorbance could be related to the concentration of analytes. The transmitted light intensity could also be used to validate that sample has covered the light beam, as indicated by a sufficiently low light level. The transmitted light measurement could also be used as a means of validating the intensity of the light source, such as when the reagent kit not present.

Referring to FIGS. 20 and 21, a reagent kit 50vi in accordance with another alternative embodiment of invention will be described. The reagent kit 50vi includes a pipette device 90 and sample display member 98, which in the current embodiment is a glass slide with a planar sample receiving surface 99. The pipette 90 has a hollow tubular body 91 which extends to an open tapered tip 92. A reagent 94, similar to the reagents described above, is disposed on the inside surface 93 of the pipette 90 adjacent the open tapered tip 92. The open tapered tip 92 is configured to draw in blood or another fluid which mixes with the reagent 94 to form a mixed sample 96 within the pipette 90. The mixed sample 96 is then transferred to the planar surface 99 of the display member 98 and the display member 98 may thereafter be positioned in the minor housing 13 of the hematofluorometer 10 and tested in a manner similar to that described above. Transfer of the sample 96 may be accomplished using a pipetter as is known or using other fluid handling equipment. The pipette 90 with reagent 94 is preferably used on a one-time basis, i.e. disposable, to ensure accurate reagent transfer, and avoiding cross contamination with other blood samples. While the illustrated display member 98 is a planar surface, the invention is not limited to such and the display member 98 may include one or more wells similar to those described in conjunction with the earlier described reagent kits.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed:

1. A reagent kit for detecting bilirubin in a fluid sample, the reagent kit comprising:
a body defining at least one fluid receiving well; and
an optical window positioned over each at least one fluid receiving well, each window formed of a material having a fluorescence intensity that is of a lower magnitude than the fluorescence to be detected from the bilirubin.

2. The reagent kit according to claim 1, wherein the optical window is of a different material than that of the body.

3. The reagent kit according to claim 2, wherein the body is made of a material that is not optimized for fluorescence measurements.

4. The reagent kit according to claim 1, wherein one or more reagents is deposited on a surface of the well or the window.

5. The reagent kit according to claim 1, wherein the body defines multiple optical measuring positions.

6. The reagent kit according to claim 5, wherein each optical measuring position is defined by a separate fluid receiving well with a corresponding optical window.

7. The reagent kit according to claim 6, wherein each fluid receiving well has an associated inlet configured to draw in fluid via capillary action.

8. The reagent kit according to claim 6, wherein adjacent fluid receiving wells are interconnected via capillary passages, and the body includes an inlet associated with one of the fluid receiving wells configured to draw in fluid via capillary action.

9. The reagent kit according to claim 5, wherein the body includes a single well and corresponding optical window, and the optical measuring positions are defined by spaced apart locations within the single well.

10. The reagent kit according to claim 9, wherein one or more of the spaced apart locations has a reagent deposited thereon.

11. The reagent kit according to claim 9, wherein the single well has an inlet passage and an outlet passage and the distance between the inlet passage and the outlet passage is significantly larger than a distance between a bottom surface of the well and the optical window.

12. The reagent kit according to claim 1, wherein each optical window is defined in a cap configured to be secured to the body over a respective one of the fluid receiving wells.

13. The reagent kit according to claim 12, wherein each cap is manufactured from the same material as the body.

14. The reagent kit according to claim 12, wherein each cap defines a through passage and the optical window is defined by a plate extending across the through passage.

15. The reagent kit according to claim 14, wherein each cap defines an internal shoulder about the through passage and the plate is spaced from the shoulder to define a receiving cavity adjacent the optical window.

16. The reagent kit according to claim 1, wherein the body is in the form of an elongated member with a cup defined on one end thereof, the cup defining the at least one fluid receiving well.

17. The reagent kit according to claim 16, wherein the cup is configured to draw fluid thereinto via capillary action.

18. The reagent kit according to claim 16, wherein a cap secured over the cup defines the optical window.

19. A hematofluorometer comprising:
an excitation source configured to generate an excitation beam,
a fluorescence detector configured to a fluorescence beam; and
a housing configured to receive a reagent kit according to claim 1 and position the reagent kit such that the excitation beam passes through one of the optical windows toward the respective well and the reflected fluorescence beam passes through the same optical window and is detected by the fluorescence detector.

20. The hematofluorometer according to claim 19, wherein the body of the reagent kit includes a light passage window opposite each optical window, and wherein the hematofluorometer further includes a light sensor within the housing configured to detect light passing through the reagent kit.

21. The hematofluorometer according to claim 20, wherein an intensity of the detected light is used to measure light absorbance of the fluid within the well.

22. The hematofluorometer according to claim 20, wherein an intensity of the detected light is used to validate that the excitation beam passed through the fluid in the well.

23. The hematofluorometer according to claim 19 further comprising a temperature sensor and processor wherein the processor is configured to correct for the temperature dependent response of the instrument or changes in the fluorescence of the reagents.

24. The hematofluorometer according to claim 19 further comprising a processor configured to correction for a hematocrit or hemoglobin content of the fluid.

25. A reagent kit comprising:
a pipette device having a hollow body extending to an open tapered tip;
a reagent disposed on an inside surface of the open tapered tip; and
a display member configured to receive and display a mixed sample from the pipette device.
